(19) United States
(12) Patent Application Publication
(10) Pub. No.: US 2004/0013696 A1
(43) Pub. Date: Jan. 22, 2004

(54) Use of ellagic acid as an anti-pollution cosmetic agent

(76) Inventors: Daniel Duche, Paris (FR); Jose Cotovio, Danmartin-en-Goele (FR); Philippe Catroux, Nogent-sur-Marne (FR)

Correspondence Address: D Douglas Price, Steptoe & Johnson, 1330 Connecticut Ave N W, Washington, DC, 20036, US

(21) Appl. No.: 10/276,540

(22) PCT Filed: May 14, 2001
(86) PCT No.: PCT/FR01/01454

(30) Foreign Application Priority Data
May 18, 2000 (FR) 0006384

(51) Int. Cl. A61K 31/366; A61K 7/00

(52) U.S. Cl. 424/401; 514/453

(57) ABSTRACT

The invention concerns the use for topical application of ellagic acid as cosmetic antipollution agent and a cosmetic treatment method for protecting the organism against pollution effects.

The present invention relates essentially to a novel use of ellagic acid, its salts, its metal complexes, its monoether or polyether, monoacyl or polyacyl derivatives and its carbonate or carbamate derivatives, derived from the hydroxyl groups, as an antipollution cosmetic agent.

Urban pollution is composed of various types of chemical products, xenobiotics and particles.

Three major categories of pollutants can exert deleterious effects on the skin and the hair: gases, heavy metals and particles that are combustion residues onto which are adsorbed a large number of organic compounds.

It is the outermost tissues that are initially and directly exposed to environmental toxic agents. The skin is directly and frequently exposed to the prooxidizing environment. The environmental sources of oxidizing agents include oxygen, solar UV radiation and also, in polluted air, ozone, nitrogen oxides and sulfur oxides. The atmospheric pollutants represented by the primary and secondary products of domestic and industrial combustion such as monocyclic and polycyclic aromatic hydrocarbons are also a major source of oxidative stress. The skin is particularly sensitive to the action of oxidative stress and the outermost layer serves as a barrier against oxidative damage. In most circumstances, the oxidizing agent is likely to be neutralized after reaction with keratin materials, but the reaction products formed may be responsible for attacks on cells and tissues.

The stratum corneum, which is the skin's barrier, is the site of contact between the air and skin tissue. The lipid/protein two-phase structure is a crucial factor of this skin barrier function. These elements can react with oxidizing agents and be impaired, which will promote the phenomena of desquamation. Ozone-induced lipid peroxidation can impair the skin in two ways:

1/ The oxidation and degradation of the lipids of the stratum corneum can impair the barrier function of the stratum corneum. Disruption of the outer lipids and of the protein architecture appear to be triggering factors in many dermatoses (psoriasis, atopic dermatitis and irritant dermatitis).

2/ The increased formation of lipid-oxidation products in the upper layers of the skin can trigger attacks in the adjacent layers of skin. The reaction of ozone (O₃) with unsaturated lipids involves addition reactions onto the double bonds. This process leads in a second stage to cleavage of the lipid chains and to the formation of aldehyde hydroperoxides and of hydrogen peroxide. This is a specific mechanism that is different than the lipoperoxidation mechanism conventionally described, which is mediated by a radical. The secondary or tertiary lipid oxidation products induced with ozone, which are less reactive than ozone that have a longer lifetime, can propagate the effect of ozone. On account of their relative stability, lipid oxidation and peroxidation products, i.e. cholesterol oxides and aldehydes, have the potential to impair cells at remote sites not directly exposed to O₃.

A significant oxidative attack on the surface layers of the stratum can initiate localized subjacent inflammatory processes, leading to the recruitment of phagocytes, which, by generating oxidizing agents, will amplify the initial oxidative processes.

In urban pollution, the concomitant exposure to O₃ and to UV can cause a synergistic oxidative stress.

Similarly, it may be thought that there is synergistic action between ozone and combustion-derived organic compounds.

Heavy metals constitute another category of pollutants.

Metal ions are required by the body in the form of trace amounts as essential nutrients. For example, several functions involving polypeptides, such as enzymatic, structural and immunological functions, require metallic cofactors.

However, other metal ions, in particular heavy metal ions when they are at nonphysiological concentrations, may impair these functions. Thus, overexposure to metals of the environment can lead to toxic effects.

Ecological studies conducted in industrialized countries show that the amounts of metals present in the atmosphere are increasing. This leads to an increase in the levels of heavy metals in body tissues following the ingestion of contaminated food and exposure to atmospheric metals.

The effects of accumulation of heavy metals may be extremely hazardous and their toxicity is partially due to the impairment of the tertiary and quaternary structures of proteins, which results in a reduction in their catalytic activity. The impaired proteins may become antigenic and bring about an immune response.

Furthermore, the accumulation of metals from pollutant particles present in the air, such as zinc, copper, cobalt, manganese, mercury or nickel, gives rise to memory disorders in children.

Another mechanism responsible for the toxic effects of metals is the competitive substitution of natural physiological cofactors with heavy metals at nonphysiological concentrations. Thus, controlling the pollutant heavy metals in the atmosphere is essential for preventing diseases in relation with exposure to the metals.

Due to the increasing contamination of the environment with heavy metals and their ubiquitous presence in the ecosystem, the skin, the hair and the accessible mucous membranes represent the largest area of contact and thus promote the accumulation of metals and their subsequent absorption into the body.

Certain metals and metal compounds present in industrial manufactured products, chemicals, jewellery, clothing, medicinal products, colorants and cleaning products are involved in primary irritation reactions, allergic reactions and carcinogenicity reactions in skin tissue.

The metals that are the main offenders in the environment are copper, cobalt, zinc, manganese, mercury, nickel and lead.

Skin rashes caused by metal-induced dermatitis are a problem encountered in people exposed to large amounts of certain metal ions. Exposure to nickel in the environment is largely due to the frequent use of this metal in jewellery articles, watch straps and clothing buttons. Sensitization to nickel with the development of dermatitis is an industrial hazard in certain occupations. The deposition of metals on the hair is an inevitable phenomenon.

The hair is a strong absorber of metals. The binding is so strong that once these bound metals have been captured by the anionic sites of the fiber, they are difficult to extract. The degree of binding of the metals to the hair generally depends on several factors, such as the size of the fiber, its porosity and the exposure time. Metals such as copper, lead and iron may interfere with chemical treatments such as the dyeing and permanent-waving of the hair.

The hair is also a preferred site for these heavy metal particles. The reason for this is that keratin fibers contain anionic sites which bind cationic heavy metals and accumulate them. Certain cosmetic products contain metals such as magnesium, copper or iron. The absorption of these metals by the keratin fibers may interfere with chemical treatments such as dyeing, bleaching or permanent-waving effects. These interactions may lead to problems in dyeing or precipitations, as described in American patent U.S. Pat. No. 5,635,167.

It has been demonstrated that certain heavy metals penetrate the skin and are accumulated (A. B. G. Landsdown. Critical Reviews in Toxicology, 1995, 25:397-462). At high concentration, they can induce: oxidation mechanisms on membrane lipids, direct cytotoxicity, liable to result in cellular necrosis and an alkylation of the cellular nucleophiles, which may be the cause of sensitization phenomena or carcinogenesis.

Another major category of pollutants consists of combustion residues in the form of particles onto which are adsorbed a large number of organic compounds, and in particular polycyclic aromatic hydrocarbons (PAHs). These polycyclic aromatic hydrocarbons adsorbed onto the surface of particles and dusts carried by urban air can penetrate skin tissue and be biotransformed therein. Their metabolism in the liver, which is well described in the literature, leads to the formation of monohydroxylated metabolites (detoxification pathway), epoxides and diol epoxides (toxifying pathway). Similar phenomena may be observed in the skin. These compounds are known to have carcinogenic and immunogenic effects on the skin.

Solutions have already been envisioned in cosmetic and therapeutic treatments by protecting tissues with compounds with sulfur-containing groups which behave like heavy metal sequestering agents, for instance the metallothioneins in patent EP 0 557 042 A1 and the amino acid compounds with sulfur-containing groups in patent application EP 0 914 815 A1.

Patent application GB 2 333 705 mentions the use of ethylenediaminedisuccinic acid in compositions for treating heavy-metal-induced skin irritations.

Moreover, document EP-A-0 496 173 describes gall-nut extracts containing ellagic acid in combination with gallic acid and hydrolyzable tannins, to prevent the harmful effects of free radicals. Said document also envisions a cosmetic application as a screening agent for protecting against ultraviolet B rays, which are responsible for ageing of the skin.

Many prior patents essentially cover the use of ellagic acid for its depigmenting, ultraviolet radiation-screening, anticancer and antiinflammatory properties.

The problem posed is thus that of protecting the skin against gases, heavy metals and organic compounds that are combustion residues and the deleterious effects thereof encountered in urban pollution, acting separately or in combination.

It has now been found, entirely surprisingly, that the use in topical application of ellagic acid, its salts, its metal complexes, its monoether or polyether derivatives, its monoacyl or polyacyl derivatives and also its carbonate or carbamate derivatives, derived from the hydroxyl groups, makes it possible to protect keratin materials, the skin and the integuments against the deleterious effects of gases, heavy metals and organic compounds that are combustion residues.

The Applicant has discovered that ellagic acid makes it possible to preserve and protect keratin materials, the skin and the integuments against the harmful effects of pollution.

Ellagic acid shows major value as a molecule that is active against the deleterious effects of pollution on the skin. It has the advantage of exerting a protective effect against pollutants of various nature at low concentrations.

Ellagic acid, also known as 2,3,7,8-tetrahydroxy-(1)benzopyrano(5,4,3-cde)(1)benzopyran-5,10-dione, is a well-known molecule belonging to the polyphenol group and is present in the plant kingdom. Reference may be made to the Merck Index 20th edition (1996), No. 3588.

Document FR-A-1 478 523 discloses a process for purifying ellagic acid and also the purified ellagic acids obtained by such a process.

Ellagic acid has the following chemical formula:

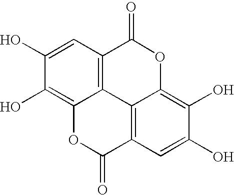

which comprises four fused rings.

Ellagic acid is commercially available, especially from the company Sigma, France.

One subject of the present invention is a use in topical application of ellagic acid, its salts, its metal complexes, its monoether or polyether derivatives, its monoacyl or polyacyl derivatives and its carbonate or carbamate derivatives, derived from the hydroxyl groups, as antipollution cosmetic agents.

The expression “antipollution cosmetic agent” means an agent that protects the skin and keratin materials so as to prevent, attenuate and/or suppress the deleterious effects of toxic gases such as ozone, metals and organic compounds that are combustion residues.

Ellagic acid and its derivatives are used as cosmetic agents for trapping toxic gases and/or as heavy-metal-chelating cosmetic agents and/or as cosmetic agents for preventing contact hypersensitivity reactions caused, inter alia, by polycyclic aromatic hydrocarbons.

A subject of the present invention is also the use of ellagic acid and its derivatives in, or for the preparation of, an antipollution cosmetic composition for topical application.

In the context of the invention, the ellagic acid salts in particular comprise the metal salts, especially of alkali metals or alkaline-earth metals, such as sodium and calcium, the amine salts such as the methyl-glutamine, diethanolamine, triethanolamine, choline and bis-triethylamine salts, the amino acid salts, especially the salts of basic amino acids such as arginine, lysine and ornithine, the metal complexes in particular comprise metal complexes with zinc and copper, and the monoacyl or polyacyl derivatives in particular comprise saturated or unsaturated acyl groups containing from 2 to 22 carbon atoms. Preferably, these acyl groups correspond to acetic acid, palmitic acid, oleic acid, linoleic acid, linolenic acid, arachidonic acid, stearic acid, brassidic acid, erucic acid, behenic acid and (all Z)-5,8,11,14,17-eicosapentaenoic acid. The abovementioned monoether or polyether derivatives are, in particular, alkoxy derivatives containing from 1 to 4 carbon atoms, or derivatives of condensation of one or more hydroxyl groups of ellagic acid with a sugar or a chain of sugars. In particular, it is 3-methoxy-ellagic acid or monoether or polyether derivatives with sugars such as glucose, arabinose, rhamnose and galactose.

The abovementioned ether or acyl derivatives may be obtained by processes for etherification or acylation of polyphenols that are well known to those skilled in the art. Some may also be obtained by extraction from plants.

The cosmetic compositions used in the invention will advantageously contain from 0.001% to 10% and preferably between 0.01% and 5% by weight of ellagic acid, its salts, its metal complexes, its monoether or polyether, monoacyl or polyacyl derivatives and its carbonate or carbamate derivatives, derived from the hydroxyl groups, relative to the total weight of the composition.

This composition may also contain at least one other antipollution compound.

Said compound may be chosen especially from anthocyans and/or derivatives thereof, compounds containing a thioether function, sulfoxides or sulfones, ergothionine and/or its derivatives, heavy-metal-chelating agents such as, for example, N,N′-dibenzylethylene-diamine-N,N′-diacetic acid derivatives, antioxidants, and cell extracts of plants from the Pontederiacea family.

The cosmetic composition used in the invention may also contain a cosmetically acceptable medium, which more particularly consists of water and/or optionally of a cosmetically acceptable organic solvent.

They may be chosen from the group consisting of hydrophilic organic solvents, amphiphilic solvents and lipophilic organic solvents, or mixtures thereof.

Among the hydrophilic organic solvents that may be mentioned, for example, are linear or branched lower monoalcohols containing from 1 to 8 carbon atoms, for instance ethanol, propanol, butanol, isopropanol and isobutanol, polyethylene glycols containing from 6 to 80 ethylene oxides, polyols such as propylene glycol, isoprene glycol, butylene glycol, glycerol, sorbitol, monoalkyl or dialkyl isosorbide, the alkyl groups of which contain from 1 to 5 carbon atoms, for instance dimethyl isosorbide, glycol ethers, for instance diethylene glycol monomethyl ether or monoethyl ether, and propylene glycol ethers, for instance dipropylene glycol methyl ether.

Amphiphilic organic solvents that may be mentioned include polyols such as propylene glycol (PPG) derivatives, such as esters of polypropylene glycol and of fatty acids, derivatives of PPG and of fatty alcohols, for instance PPG-23 oleyl ether and PPG-36 oleate.

Lipophilic organic solvents that may be mentioned, for example, include fatty esters such as diisopropyl adipate, dioctyl adipate and alkyl benzoates.

The organic solvents are preferably chosen from monofunctional or polyfunctional alcohols, optionally oxyethylenated polyethylene glycols, polypropylene glycol esters, sorbitol and its derivatives, dialkyl isosorbides, glycol ethers and polypropylene glycol ethers, and fatty esters.

The organic solvents may represent from 5% to 98% of the total weight of the composition.

In order for the compositions used in the invention to be more pleasant to use, softer to apply, more nourishing and more emollient, it is possible to add a fatty phase to the medium of these compositions.

The fatty phase preferably represents from 0 to 50% relative to the total weight of the composition.

This fatty phase may comprise one or more oils preferably chosen from the group consisting of:

volatile or nonvolatile, linear, branched or cyclic, organomodified or non-organomodified, water-soluble or liposoluble silicones,

mineral oils such as liquid paraffin and liquid petroleum jelly,

oils of animal origin such as perhydrosqualene,

oils of plant origin such as sweet almond oil, avocado oil, castor oil, olive oil, jojoba oil, sesame oil, groundnut oil, macadamia oil, grapeseed oil, rapeseed oil or coconut oil,

synthetic oils such as purcellin oil and isoparaffins,

fluoro oils and perfluoro oils,

fatty acid esters such as purcellin oil.

Said fatty phase may also comprise as fatty substances one or more fatty alcohols, fatty acids or waxes (paraffin wax, polyethylene wax, carnauba wax or beeswax).

In a known manner, the compositions used in the invention may also contain adjuvants that are common in cosmetics, such as standard aqueous or lipophilic gelling agents and/or thickeners, hydrophilic or lipophilic active agents, preserving agents, antioxidants, fragrances, emulsifiers, moisturizers, pigmenting agents, depigmenting agents, keratolytic agents, vitamins, emollients, sequestering agents, surfactants, polymers, acidifying or basifying agents, fillers, free-radical scavengers, ceramides, sunscreens, especially ultraviolet screening agents, insect repellents, slimming agents, dyestuffs, bactericides and antidandruff agents.

The amounts of these various adjuvants are those conventionally used in the fields under consideration.

Needless to say, a person skilled in the art will take care to select the optional compound(s) to be added to the composition according to the invention, such that the advantageous properties intrinsically associated with the composition in accordance with the invention are not, or are not substantially, adversely affected by the envisioned addition.

The compositions used according to the invention may be in any presentation form normally used for topical application, especially in the form of an aqueous, aqueous-alcoholic or oily solution, an oil-in-water or water-in-oil or multiple emulsion, an aqueous or oily gel, a liquid, pasty or solid anhydrous product or a dispersion of oil in an aqueous phase using spherules, these spherules possibly being polymer nanoparticles such as nanospheres and nanocapsules, or better still lipid vesicles of ionic and/or nonionic type.

The compositions used in the present invention may be more or less fluid and may have the appearance of a white or colored cream, an ointment, a milk, a lotion, a serum, a paste, a mousse or a solid.

They may optionally be applied to the skin in aerosol form.

They may also be applied in solid form, and for example in the form of a stick.

They may be used as care products and/or as makeup products.

The compositions according to the invention may have a pH of between 3 and 8 and preferably between 5 and 7.

Another subject of the invention consists of a cosmetic treatment process for protecting the body against the effects of pollution, which consists in applying to the skin a cosmetically effective amount of ellagic acid, its salts, its complexes, its monoether or polyether, monoacyl or polyacyl derivatives and its carbonate or carbamate derivatives, derived from the hydroxyl groups.

Another cosmetic treatment process according to the invention, for protecting the body against the effects of pollution, consists in applying to the skin a cosmetic composition according to the invention, as defined above.

The examples that follow are intended to illustrate the invention without, however, being limiting in nature.

Experiments

1. Protection Against the Effects of Ozone

Principle:

Ozone has the capacity to oxidize cell constituents, especially generating carbonylated proteins and lipid hydroperoxides. Quantification of the lipid hydroperoxides is one means of measuring the oxidative stress induced by an exposure of skin tissue to this pollutant. A decrease in their content indicates a protective effect of ellagic acid.

Cell Type and Culture:

The study was performed on a monolayer culture of human keratinocytes obtained from plastic surgery. The cells are inoculated on D-3 into 48-well dishes at a rate of 25 000 cells/cm² in 500 μl of culture medium. The incubations are performed at 37° C., 5% CO₂ in humid atmosphere.

Pretreatment of the Keratinocytes with Ellagic Acid (D-1):

The cells were pretreated for 24 hours with ellagic acid (100 μM final).

Incorporation of an Oxidative Stress Marker, DCFH-DA (2,7-dichlorofluorescin diacetate):

Hydroperoxides constitute an intracellular stress marker. They are detected and quantified by means of a fluorescence technique (Lebel C. P., Ischiropoulos H. and Bondy S. C. (1992) Evaluation of the probe 2,7-di-chlorofluorescin as an indicator of Reactive Oxygen Species formation and oxidative stress. Chem. Res. Toxicol.: 5: 227-231).

In the presence of intracellular hydroperoxides and peroxidases, DCFH is oxidized to fluorescent 2,7-di-chlorofluorescein (DCF).

The cells, pretreated for 24 hours with ellagic acid, are then washed with phosphate-buffered saline (PBS) and placed in contact for 30 minutes with a solution of DCFH-DA (500 μl/well), prepared in the culture medium to a concentration of 320 μM.

Exposure to Ozone:

The cells are again rinsed with PBS buffer and then placed in contact with an ellagic acid solution (100 μl/well), prepared in PBS to a concentration of 200 μM. They are then exposed to ozone (10 ppm), in a humid atmosphere, in an incubator set at 37° C.

Measurement of Ozone-Induced Lipid Hydroperoxides:

The formation of fluorescent DCF (excitation screen at 485 nm and emission screen at 530 nm) resulting from the production of hydroperoxides is measured after different times of exposure to ozone: 0, 5, 10 and 20 minutes.

Results:

Toxicity of ozone toward human keratinocytes in culture, in the absence and presence of ellagic acid at a concentration of 200 μM, as a function of the exposure time.

Fluorescence observed in the presence of ellagic acid,
expressed as % relative to the unprotected controls for
each time

| 5 min. of contact | 10 min. of contact | 20 min. of contact |
|---|---|---|
| % fluorescence | % fluorescence | % fluorescence |
| observed | observed | observed |
| ± SEM | ± SEM | ± SEM |
| 28.2 ± 3.3 | 36.3 ± 3.4 | 52.3 ± 4.4 |

For each time, the fluorescence values of the unprotected controls are set at 100%. The resulting values in the presence of ellagic acid are then expressed relative to this control value. Ellagic acid significantly decreases ozone-induced stress. This protection is at a maximum from 5 minutes of exposure onward (71.8% drop in induced stress). It is still significant after 20 minutes of exposure (47.7% drop in induced stress).

Starting with a biological model, in vitro, using human keratinocytes in culture, we have shown:

that a representative agent of a category of atmospheric pollutants such as ozone leads under our experimental conditions to the appearance of substantial stress,

that ellagic acid exerts a highly significant protective effect against the effect induced by this pollutant.

2. Protection Against the Cytotoxicity of Heavy Metals

Principle:

Heavy metals such as cadmium, nickel, lead, mercury, etc. exert a cytotoxic effect on the cells of various organs, including the skin. The technique for measuring the cell viability via the neutral red incorporation test made it possible to demonstrate the cytoprotective effect of ellagic acid against the toxicity of cadmium.

Inoculation of the Cells and Culture Conditions:

The study was performed on a monolayer culture of human keratinocytes obtained from plastic surgeries. The cells are inoculated on D-3 into 96-well dishes at a rate of 25 000 cells/cm² in 100 μl of culture medium. The incubations are performed at 37° C. in a humid atmosphere enriched with 5% CO₂.

Treatment of the Cells:

Initially, the cells are treated for 24 hours with increasing concentrations (0, 10, 25, 50, 75, 100, 150 and 200 μM) of cadmium chloride (CdCl₂), so as to determine its cytotoxicity. In a second stage, they are also treated for 24 hours with the same concentrations of CdCl₂ in the presence of ellagic acid (200 and 100 μM, concentrations corresponding to the maximum dose and half-maximal dose of ellagic acid that are noncytotoxic to the cells).

Measurement of the Cell Viability:

At the end of the treatment, the cell viability is determined by means of the neutral red incorporation test (POS 55/006) and reading at 550 nm (ref: Borenfreund, E and Puerner, J. A. (1984) A simple quantitative procedure using monolayer cultures for cytotoxicity assays. Tissue Culture Methods; 9: 7-9).

The cells are rinsed with PBS buffer in order to remove the treatment solutions, and are then incubated for three hours at 37° C. in a neutral red solution (100 μl) prepared to a concentration of 0.5 mg/ml in the culture medium. They are then rinsed with PBS buffer and then fixed for one minute in a formaldehyde/calcium solution. The neutral red is then extracted with an ethanol/acetic acid solution (100 μl/well). The amount extracted is determined by reading the optical density on a spectrophotometer at 550 nm.

The concentration of CdCl₂ that induces a 50% drop in the cell viability (IC-50) is then calculated.

Results:

Cytotoxicity of cadmium chloride toward human keratinocytes in culture, in the absence and presence of ellagic acid at two concentrations, 100 and 200 μM (n=4).

IC-50 of cadmium chloride

| | |
|---|---|
| | With ellagic acid |
| Without ellagic acid | Mean ± SEM |

| Mean ± SEM | 100 μM | 200 μM |
|---|---|---|
| 39 ± 1.2 μM | 99 ± 2.9 μM | 167 ± 9.8 μM |

The cadmium chloride alone shows substantial toxicity, with an IC-50 of 39 μM. In the presence of ellagic acid, the cytotoxicity of cadmium chloride decreases greatly (which corresponds to an increase in the IC-50):

at 100 μM of ellagic acid, the cytotoxicity decreases by a factor of 2.5,

at 200 μM of ellagic acid, the cytotoxicity decreases by a factor of 4.3.

Starting with a biological model in vitro, using human keratinocytes in culture, we have shown:

that a representative agent of a category of atmospheric pollutants (heavy metals) such as cadmium leads under our experimental conditions to high toxicity,

that ellagic acid exerts a cytoprotective effect against the toxicity of this pollutant.

3. Protection Against the Alkylation of Nucleophiles Induced by the Cutaneous Metabolism of Polycyclic Aromatic Hydrocarbons (PAHs)

Principle:

Polycyclic aromatic hydrocarbons (PAHs), adsorbed onto the surface of particles and dust conveyed by the urban atmosphere, can penetrate into skin tissue and be biotransformed therein. Their metabolism in the liver, which is well-described in the literature, leads to the formation of monohydroxylated metabolites (detoxification pathway), epoxides and diol epoxides (toxifying pathway). It follows the same profile in the skin. The toxifying pathway of the epoxides and diol epoxides gives rise to an alkylation of nucleophiles (proteins and DNA), which it is possible to demonstrate by measuring the covalent binding to these macromolecules by studying the metabolism of a radiolabeled (¹⁴C-benzo-[a]pyrene) PAH. After consequent or repeated exposures to the pollutant, its potential toxicity can lead to a contact hypersensitivity in the case of the alkylation of proteins.

The principle of the study was to demonstrate a protective effect of ellagic acid toward alkylating reactive species produced by the cutaneous metabolism of radiolabeled B[a]P by measuring the radioactivity covalently bound to the proteins.

Cell Type Studied and Culture:

The study was performed on a monolayer culture of human keratinocytes obtained from plastic surgeries. The cells are inoculated on D-3 into 6-well dishes at a rate of 53 000 cells/cm². The incubations are performed at 37° C. in humid atmosphere enriched with 5% CO₂.

Treatment of the Cells:

The cells are placed in contact for 24 hours with ¹⁴C-B[a]P (20 μM) and ellagic acid from Sigma, France (100 μM), both coincubated. After this contact, the cells were washed with PBS buffer, scraped in the same buffer (0.5 ml) and then frozen in liquid nitrogen and stored at −80° C. until the analysis.

Measurement of the Covalent Binding to Cell Proteins:

The covalent binding to cell proteins is measured according to the protocol of Hoellinger et al., adapted to a filtration method in 96-well microplates (H. Hoellinger, M. Sonnier, J. Gray, T. A. Connors, J. Pichon and N. H. Nam. In vitro covalent binding of cismethrin, bioresmethrin and their common alcohol to hepatic proteins. Toxicol. Appl. Pharmacol., 1985, 77, 11-18).

An aliquot fraction of the cell proteins (200 μl) is precipitated in the microplate wells with 10% perchloric acid (50 μl). The plate is then transferred to a filtration system under vacuum, in which the contents of the wells are taken up by suction and the proteins retained on the filter membranes, washed with the solvents ethyl acetate (3×200 μl), acetone (200 μl), ethanol (200 μl) and PBS buffer (200 μl). The aim of this series of washes is to remove from the proteins any radioactivity not covalently bound. The microplate filters are recovered individually and the proteins retained at their surface are digested in 1N sodium hydroxide (400 μl) for 24 hours at 37° C. Samples are then taken to assay the proteins and count the radioactivity of the solution.

The results are expressed as nmol of B[a]P bound per mg of proteins.

Results:

Covalent binding of benzo(a)pyrene to human keratinocytes in culture in the absence and presence of ellagic acid at a concentration of 100 μM (n=4).

Covalent binding of B (a) P

| Without ellagic acid | With ellagic acid |
|---|---|
| in nmol of B (a) P per | in nmol of B (a) P per |
| mg of protein | mg of protein |
| Mean ± SEM | Mean ± SEM |
| 0.28 ± 0.07 μM | 0.09 ± 0.02 μM |

Benzo(a)pyrene alone at a concentration of 20 μM shows considerable reactivity with a covalent binding to keratinocyte proteins of 0.28 nmol of B(a)P per mg of protein. In the presence of ellagic acid, the reactivity of B(a)P decreases greatly (reduction in the reactivity by a factor of 3.1).

Starting with a biological model in vitro using a cell of human epithelial origin (keratinocyte/skin), we have shown:

that a representative agent of a category of atmospheric pollutants (PAH) such as benzo[a]pyrene leads under our experimental conditions to a potential toxicity associated with its metabolic capacities to produce alkylating species,

that ellagic acid exerts a protective effect against this form of toxicity induced by such pollutants.

### FORMULATION EXAMPLES

### Example 1

According to the usual preparation techniques, the constituents below are mixed together to prepare an emulsion.

COMPOSITION FOR TOPICAL APPLICATION

| | | |
|---|---|---|
| sodium salt of ellagic acid | 5 | g |
| polyethylene glycol oxyethylenated with | 3 | g |
| 50 mol of ethylene oxide | | |
| monodiglyceryl stearate | 3 | g |
| liquid petroleum jelly | 24 | g |
| cetyl alcohol | 5 | g |
| water | qs 100 | g |

### Example 2

In the same manner, an emulsion is prepared according to a standard technique, using the following compounds:

| | | |
|---|---|---|
| diethanolamine salt of ellagic acid | 1 | g |
| octyl palmitate | 10 | g |
| glyceryl isostearate | 4 | g |
| purcellin oil | 23 | g |
| vitamin E | 1 | g |
| glycerol | 3 | g |
| water | qs 100 | g |

### Example 3

In the same manner, an emulsion is prepared according to a standard technique, using the following compounds:

| | | |
|---|---|---|
| 3-methoxyellagic acid | 0.01 | g |
| octyl palmitate | 10 | g |
| glyceryl isostearate | 4 | g |
| liquid petroleum jelly | 20 | g |
| sorbitol | 2 | g |
| vitamin E | 1 | g |
| glycerol | 3 | g |
| water | qs 100 | g |

### Example 4

In the same manner, an emulsion is prepared according to a standard technique, using the following compounds:

| | | |
|---|---|---|
| monoacetyl ellagic acid | 0.5 | g |
| octyl palmitate | 10 | g |
| glyceryl isostearate | 4 | g |
| liquid petroleum jelly | 24 | g |
| vitamin E | 1 | g |
| glycerol | 3 | g |
| water | qs 100 | g |

### Example 5

Starting with the constituents below, the following composition is formulated:

| | | |
|---|---|---|
| calcium salt of ellagic acid | 1.5 | g |
| jojoba oil | 13 | g |
| methyl isopropyl para-benzoxybenzoate | 0.05 | g |
| potassium sorbate | 0.3 | g |
| cyclopentadimethylsiloxane | 10 | g |
| stearyl alcohol | 1 | g |
| stearic acid | 4 | g |
| polyethylene glycol stearate | 3 | g |
| vitamin E | 1 | g |
| glycerol | 3 | g |
| water | qs 100 | g |

### Example 6

Starting with the constituents below, the following composition is formulated:

| | | |
|---|---|---|
| ellagic acid complexed with zinc | 1 | g |
| jojoba oil | 13 | g |
| methyl isopropyl para-benzoxybenzoate | 0.05 | g |
| potassium sorbate | 0.3 | g |
| cyclopentadimethylsiloxane | 10 | g |
| stearyl alcohol | 1 | g |
| N,N′-bis (3-hydroxybenzyl) ethylene | 0.01 | g |
| diamine-N,N′-diacetic acid | | |
| stearic acid | 4 | g |
| polyethylene glycol stearate | 3 | g |
| vitamin E | 1 | g |
| glycerol | 3 | g |
| water | qs 100 | g |

### Example 7

Starting with the constituents below, the following composition is formulated:

| | | |
|---|---|---|
| choline salt of ellagic acid | 0.5 | g |
| jojoba oil | 13 | g |
| methyl isopropyl para-benzoxybenzoate | 0.05 | g |
| potassium sorbate | 0.3 | g |
| cyclopentadimethylsiloxane | 10 | g |
| stearyl alcohol | 1 | g |
| stearic acid | 4 | g |
| cell extract of water hyacinth | 0.05 | g |
| polyethylene glycol stearate | 3 | g |
| vitamin E | 1 | g |
| glycerol | 3 | g |
| water | qs 100 | g |

The cell extract of water hyacinth (Eichhornia crassipes) was obtained by this process: 12 water hyacinth stems were washed with water and then crudely drained. After treating in a knife mill (chopping processor), 700 g of ground material were obtained. Addition of 700 ml of H₂O and then 300 ml of MilliQ H₂O. Further treatment in the chopping processor for five minutes, centrifugation for 20 minutes at 8 000×G, Whatmann GFD and then GFF filtration and freeze-drying: 5.43 g of lyophilizate are thus obtained.

1. The use in topical application of ellagic acid, its salts, its metal complexes, its monoether or polyether, monoacyl or polyacyl derivatives and its carbonate or carbamate derivatives, derived from the hydroxyl groups, as an antipollution cosmetic agent.

2. The use in topical application of ellagic acid, its salts, its metal complexes, its monoether or polyether, monoacyl or polyacyl derivatives and its carbonate or carbamate derivatives, derived from the hydroxyl groups, as a cosmetic agent for trapping toxic gases and/or as a heavy-metal-chelating cosmetic agent and/or as a cosmetic agent for preventing the contact hypersensitivity caused by polycyclic aromatic hydrocarbons.

3. The use of ellagic acid, its salts, its metal complexes, its monoether or polyether, monoacyl or polyacyl derivatives and its carbonate or carbamate derivatives, derived from the hydroxyl groups in, or for the preparation of, an antipollution cosmetic composition for topical application.

4. The use as claimed in one of claims 1 to 3, characterized in that the ellagic acid salts comprise a metal salt, in particular of an alkali metal or alkaline-earth metal, such as sodium and calcium, the amine salts such as the methylglutamine, diethanolamine, triethanolamine, choline and bis-triethylamine salts, the amino acid salts, especially the salts of basic amino acids such as arginine, lysine and ornithine, the metal complexes with zinc and copper and the monoacyl or polyacyl derivatives comprising saturated or unsaturated acyl groups containing from 2 to 22 carbon atoms. Preferably, these acyl groups correspond to acetic acid, palmitic acid, oleic acid, linoleic acid, linolenic acid, arachidonic acid, stearic acid, brassidic acid, erucic acid, behenic acid and (all Z)-5,8,11,14,17-eicosapentaenoic acid. The abovementioned monoether or polyether derivatives are alkoxy derivatives containing from 1 to 4 carbon atoms, or derivatives of condensation of one or more hydroxyl groups of ellagic acid with a sugar or a chain of sugars, in particular 3-methoxyellagic acid or its monoether or polyether derivatives with sugars such as glucose, arabinose, rhamnose and galactose.

5. The use as claimed in claim 3 or 4, characterized in that said antipollution cosmetic composition contains from 0.001% to 10% and preferably between 0.01% and 5% by weight of ellagic acid, its salts, its metal complexes, its monoether or polyether, monoacyl or polyacyl derivatives and its carbonate or carbamate derivatives, derived from the hydroxyl groups, relative to the total weight of the composition.

6. The use as claimed in one of claims 3 to 5, characterized in that said composition also contains at least one other antipollution compound.

7. The use as claimed in claim 6, characterized in that said compound is chosen from the group consisting of anthocyans and/or derivatives thereof, compounds containing a thioether function, sulfoxides or sulfones, ergothionine and/or its derivatives, heavy-metal-chelating agents such as, for example, N,N′-dibenzylethylenediamine-N,N′-di-acetic acid derivatives, antioxidants, and cell extracts of plants from the Pontederiacea family.

8. The use as claimed in any one of claims 1 to 7, characterized in that said composition may also contain a cosmetically acceptable medium consisting of water and/or optionally of a cosmetically acceptable organic solvent.

9. The use as claimed in claim 8, characterized in that the organic solvent is chosen from the group consisting of hydrophilic organic solvents, amphiphilic solvents and lipophilic organic solvents, or mixtures thereof.

10. The use as claimed in any one of claims 8 to 10, characterized in that the organic solvents are preferably chosen from monofunctional or polyfunctional alcohols, optionally oxyethylenated polyethylene glycols, polypropylene glycol esters, sorbitol and its derivatives, dialkyl isosorbides, glycol ethers, polypropylene glycol ethers and fatty esters.

11. The use as claimed in claim 8 or 10, characterized in that the organic solvent(s) represent(s) from 5% to 98% of the total weight of the composition.

12. The use as claimed in any one of claims 1 to 11, characterized in that said composition also comprises at least one fatty phase.

13. The use as claimed in claim 12, characterized in that the fatty phase represents from 0 to 50% relative to the total weight of the composition.

14. The use as claimed in any one of claims 1 to 13, characterized in that said composition also contains at least one additive chosen from the group consisting of standard aqueous or lipophilic gelling agents and/or thickeners, hydrophilic or lipophilic active agents, preserving agents, antioxidants, fragrances, emulsifiers, moisturizers, pigmenting agents, depigmenting agents, keratolytic agents, vitamins, emollients, sequestering agents, surfactants, polymers, acidifying or basifying agents, fillers, free-radical scavengers, ceramides, sunscreens, especially ultraviolet screening agents, insect repellents, slimming agents, dyestuffs, bactericides and antidandruff agents.

15. The use as claimed in any one of claims 1 to 14, characterized in that said composition is in the form of an aqueous, aqueous-alcoholic or oily solution, an oil-in-water or water-in-oil or multiple emulsion, an aqueous or oily gel, a liquid, pasty or solid anhydrous product or a dispersion of oil in an aqueous phase using spherules.

16. The use as claimed in any one of claims 1 to 15, characterized in that said composition has the appearance of a white or colored cream, an ointment, a milk, a lotion, a serum, a paste, a mousse or a solid.

17. A cosmetic treatment process for protecting the body against the effects of pollution, characterized in that it consists in applying to the skin a cosmetically effective amount of ellagic acid, its salts, its complexes, its monoether or polyether, monoacyl or polyacyl derivatives and its carbonate or carbamate derivatives, derived from the hydroxyl groups.

18. A cosmetic treatment process for protecting the body against the effects of pollution, characterized in that it consists in applying to the skin a composition as defined in any one of claims 1 to 16.
